(19) United States
(12) Patent Application Publication
(10) Pub. No.: US 2011/0123590 A1
(43) Pub. Date: May 26, 2011

(54) Selective Expansion of Regulatory T Cells

(75) Inventors: Makio Iwashima, River Forest, IL (US); Nagendra Singh, Augusta, GA (US)

(73) Assignee: Medical College of Georgia Research Institute, Inc .

(21) Appl. No.: 12/781,536

(22) Filed: May 17, 2010

Related U.S. Application Data
(63) Continuation of application No. PCT/US2008/083331, filed on Nov. 13, 2008.
(60) Provisional application No. 61/004,006, filed on Nov. 21, 2007.

(51) Int. Cl. A61K 9/00; C12N 5/02; C12N 5/071; C12M 3/00; A61K 35/26

(52) U.S. Cl. 424/422; 435/395; 435/372.3; 435/289.1; 424/93.71; 435/325

(57) ABSTRACT

Methods for expanding regulatory T cells in vitro are provided. It has been discovered that regulatory T cells can be expanded in vitro by culturing a mixed population of lymphocytes on planar cell culture substrates, for example cell culture dishes, coated with binding partners for TCR complex and CD28. Remarkably, culturing mixed populations of lymphocytes on planar substrates coated with anti-CD3 and anti-CD28 antibodies induced apoptosis of effector T cells. It is believe that this is the first cell culture technique that expands regulatory T cells while inducing the apoptosis of effector cells in the mixed lymphocyte population using binding partners for cell surface proteins immobilized on planar substrate. Because effector T cells are typically present in mixed populations of lymphocytes, effector T cells compete with regulatory T cells and typically overtake the cell culture. The disclosed methods decrease the population of effector cells allowing regulatory T cells to expand by at least 100, typically by 400 fold. The resulting cultures of regulatory T cells have less than about 20%, 15%, or 10% of effector T cells.

### CROSS-REFERENCE TO RELATED APPLICATIONS

This application claims benefit of and priority to U.S. Provisional Patent Application No. 61/004,006 filed on Nov. 21, 2007.

### FIELD OF THE INVENTION

Embodiments of the invention are generally directed to methods of culturing cells, in particular to methods for the selective expansion of regulatory T cells.

### BACKGROUND OF THE INVENTION

Regulatory T cells (Tregs) are a subset of T cells that are now increasingly appreciated for their role in immunological defense. Described in the early 1970s by Gershon and Kondo as T cells that were able to suppress immune responses, they were given the early name of suppressor T cells (Gershon and Kondo, Immunology, 18:723-37 (1970)). At that time it was believed that these suppressor T cells were able to mediate their function by secreting antigen-specific factors (Gershon et al., J Immunol. 108: 586-90 (1972)). However, as research progressed, the inability to isolate and/or demonstrate the function of the suppressor T cells ultimately led to their downfall, with some researchers going so far as to question their existence. The interest in suppressor T cells was revived in 1995 when a Japanese group led by Dr. Sakaguchi demonstrated that a small sub-population of T cells (˜10% of the total CD4 T cell population) expressing the interleukin (IL)-2 receptor α-chain (CD25) was directly responsible for preventing autoimmune disease in mice; thus, Balb/c nu/nu mice (abnormal thymus) that adoptively received a CD4 T cell pool deficient of the CD4+CD25+ T cell subset spontaneously developed autoimmune diseases, such as thyroiditis, gastritis and adrenalitis in a dose dependent fashion (Sakaguchi, S. et al., J Immmunol, 155:1151-64 (1995)). This newly defined sub-population of CD4+CD25+ T cells, whose existence has been confirmed by other research groups, has opened new avenues of research in a vast array of medical disorders, including host versus graft rejection, infections, cancer and autoimmune diseases (Coleman, C. et al., J. Cell. Mol. Med. 11(6):1291-1325 (2007)). Tregs maintain immunological self tolerance, preventing autoimmunity. Tregs also control immune responses to tumors, infections, allergens, and transplants.

Initially, thymic-derived Tregs were identified as a CD4+CD25+ T cell population with immunosuppressive properties that constitute 5-10% of the total peripheral CD4+ T cells (Sakaguchi, S., Cell, 101(5):455-458 (2000)). Later, Tregs were identified as FoxP3+T cells (Fontenot, J. D., et al,. Immunity 22:329-341 (2005)). Thus, naturally occurring CD4+CD25+FoxP3+ T cells represent regulatory T cells, while CD4+CD25+FoxP3− T cells constitute effector T cells. It is important to note that upon activation all the CD4+CD25− T cells express CD25 on cell surface. In human and rats CD8+FoxP3+ regulatory T cells have also been identified. In other words irrespective of CD25, CD4 or CD8 expression, FoxP3 identifies Tregs, while FoxP3 negative cells represent the effector T cells.

Using Tregs to manipulate immune responses has been an area of intense research. Unfortunately, the use of Tregs as a therapeutic or in scientific research typically requires millions of cells. Because Tregs make up such a small percentage of T cells, harvesting millions of them is extremely difficult. Thus, methods for expanding Tregs in culture are needed.

Recent reports have shown that Tregs proliferate and retain their antigen-dependent suppressive functions when cultured with antigen presenting cells (APCs), particularly antigen-loaded mature dendritic cells (DCs). When Tregs specific for a pancreatic islet cell antigen are stimulated by DCs together with IL−2, the expanded antigen-specific T cells regulate the development of autoimmune diabetes in nonobese diabetic mice and do so much more effectively than polyclonal populations (Yamazaki et al., PNAS, 103(8):2758-2763 (2006)).

At present, the most popular and widely used technique for expansion of Tregs uses anti-CD3 and anti-CD28 coated beads to stimulate T cells in culture (Tang, Q., J. Exp. Med, 199:1455 (2004); Clark et al., DYNAlogue, 16-17 (2003)). Unfortunately, this technique expands effector T cells better than regulatory T cells (Tang, Q., J Exp. Med, 199:1455 (2004)). Additionally and more importantly, it does not actively eliminate effector T cells that progressively overwhelm the culture. Successful expansion of regulatory T cells by beads coated anti-CD3 and anti-CD28 require>98% of purity of CD4+CD25+CD62L+ cells in the initial seed of cells (Tang, Q., J. Exp. Med, 199:1455 (2004)).

Thus, it is an object of the invention to provide improved methods for expanding Tregs in culture.

It is another object to provide cell cultures enriched with Tregs.

It is still another object to provide cell culture substrates for expanding Tregs.

It is another object to provide implants for expanding Tregs in vivo.

It is another object to provide methods of treatment using expanded Tregs.

### SUMMARY OF THE INVENTION

Methods for expanding regulatory T cells in vitro are provided. It has been discovered that regulatory T cells (FoxP3+ cells) can be expanded in vitro by culturing a mixed population of lymphocytes on a planar cell culture substrate, for example cell culture dishes, coated with binding partners for T cell receptor (TCR) complex (e.g., anti-CD3 antibody) and CD28 (e.g., anti-CD28 antibody). Remarkably, culturing mixed populations of lymphocytes on planer substrates coated with anti-CD3 and anti-CD28 antibodies induced apoptosis of effector T cells (FoxP3−). It is believed that this is the first cell culture technique that expands regulatory T cells while inducing the apoptosis of effector cells in the mixed lymphocyte population using binding partners for cell surface TCR and CD28, for example anti-CD3 and anti-CD28 antibodies. Because effector T cells are typically present in mixed populations of lymphocytes, effector T cells compete with regulatory T cells and due to faster growth typically overtake the cell culture. The disclosed methods decrease the population of effector T cells in Treg expansion cultures. Even when cultures are started with total CD4 T cells (˜10% Tregs and remaining ˜90% are effector T cells) after 1-2 weeks of expansion >70% of the T cells are Tregs.

One embodiment provides a method for expanding regulatory T cells by culturing the T cell populations containing regulatory T cells on a planar substrate having binding partners for the TCR complex and CD28. Exemplary binding partners for the TCR complex include anti-CD3 antibodies, anti-TCR-β antibodies, and MHC-peptide tetramers. Exemplary binding partners for CD28 include anti-CD28 antibodies or B-7 molecules. The binding partners are attached to the substrate in an amount effective to expand FoxP3+ T cells (both CD4+ or CD8+) and promote apoptosis of FoxP3− T cells (both CD4+ or CD8+). The binding partners for the TCR complex and CD28 are independently selected from the proteins, that bind the TCR complex and CD28. The disclosed methods can be used to expand mammalian regulatory T cells including human regulatory T cells.

Another embodiment provides a method for enriching a T cell culture with FoxP3+ cells by culturing the T cells on a planar substrate having binding partners for the TCR and CD28 in an amount effective to expand FoxP3+ T cells and induce apoptosis of FoxP3− T cells. Typically, the cells are cultured for about 4 days to about two weeks. In one embodiment the cells are cultured for about 7 or about 8 days.

Still another embodiment provides a cell culture vessel having a substrate, wherein ligands for TCR complex and CD28 (as above) are attached to the substrate in an amount effective to expand FoxP3+ T cells) and promote apoptosis of Foxp3− T cells. Preferably the cell culture vessel has a substantially planar substrate or any other device that would force constant interaction of coated ligands with TCR complex and CD28 on T cells.

Yet another embodiment provides a method for treating one or more symptoms of a harmful inflammatory or autoimmune disease or disorder by administering a subject to an effective amount of the regulatory T cells obtained by the disclosed methods to inhibit or reduce an immune response in the subject.

Another embodiment provides using a implanted device coated with ligands for TCR complex and CD28 in order to expand antigen specific regulatory T cells to induce apoptosis of effector T cells for purpose of inhibiting or reducing an harmful immune responses or autoimmune diseases in the subject.

### BRIEF DESCRIPTION OF THE DRAWINGS

FIGS. 1A-F are schematic diagrams showing possible methods of stimulating T cells in culture with TCR complex ligand (anti-CD3) and CD28 ligand (anti-CD28).

FIG. 1A shows beads (curved substrate) coated with anti-CD3 and anti-CD28 antibodies stimulating a resting T cell (Bead-bound stimulation).

FIG. 1B shows anti-CD3 and anti-CD28 antibodies immobilized on a planar substrate stimulating resting T cells (Plate-bound stimulation).

FIG. 1C shows anti-CD3 antibody attached to the surface of a culture vessel and anti-CD28 antibody suspended in the culture media.

FIG. 1D shows the arrangement of FIG. 1A after a few days (3-4) by which the cells have had a chance to contact the antibodies.

FIG. 1E shows the arrangement of FIG. 1B after the cells have had a chance to contact the antibodies.

FIG. 1F shows the arrangement of FIG. 1C after a few days (3-4) by which the cells have had a chance to contact the antibodies. The arrows indicate anti-CD28 and anti-CD3 antibodies respectively.

FIG. 2 shows a line graph of cpm (×1000) versus time (hours) indicative of proliferation of CD4+CD25− (open squares) and CD4+CD25+ (closed squares) T cells induced by plate bound anti-CD3 and anti-CD28 antibody in the presence of IL−2. Proliferation of each sample was measured by ³H-thymidine uptake (cpm (×1000)) for 12 hours after the point indicated at x-axis.

FIG. 3A shows a line graph of expansion of cells (in millions, Y axis) versus days of culture by plate bound technique. CD4+CD25+ cells are shown in filled rectangle and CD4+CD25− cells are shown in open rectangle.

FIG. 3B shows a line graph of expansion of CD4+CD25− (open rectangle) and CD4+CD25+ cells (filled triangle) (in millions, Y axis) versus days of cell culture by the bead bound technique.

FIGS. 4A-D show dot plot of annexin-V and 7-Amino-actinomycin D (7-AAD) staining of CD4+CD25− and CD4+CD25+ T cells 5 days after stimulation with plate bound (FIGS. 4A and 4B) or bead bound conditions (FIGS. 4C and 4D) in the presence of IL−2. The numbers within the plot represent the percent positive cells in the corresponding quadrant. Only cells in lower left quadrant are live cells, while in lower right are in the process of apoptotic death. Cells in the upper left and right quadrant are dead cells.

FIGS. 5A-D show histogram plots of FoxP3 expression by CD4+CD25− and CD4+CD25+ T cells after their expansion with plate bound (FIGS. 5A and 5B) or bead bound (FIGS. 5C and 5D) stimulations in the presence of IL−2 for 8 days. The numbers within the plot represent the percent positive cells in the corresponding gate.

FIGS. 6A and B show histogram plots of anti-CD3 induced proliferation of CD4+CD25− T cells (freshly isolated from mouse spleen) and plate bound expanded CD4+CD25+ T cells (FIG. 6A). In FIG. 6B proliferation of CD4+CD25− T cells alone or in combination with plate bound expanded CD4+CD25+ T cells are shown.

FIG. 7 shows histogram plots of FoxP3 expression by total CD4 T cells on day 0 or 7 days after culture with either plate bound or bead bound stimulation in the presence of IL−2. The numbers within the plot represent the percent positive cells in the corresponding gate.

FIG. 8A shows a histogram plot of fold expansion of FoxP3+ or FoxP3− cells after 11 days of plate bound stimulation of total mouse CD4+ T cells in the presence of IL−2.

FIG. 8B shows a line graph of suppression of proliferation of CD4+CD25− (cpm (×1,000)) by expanded Treg (▴) as in FIG. 8A, and fresh Tregs ().

FIG. 8C shows a line graph of percent weight change versus days in in immunodeficient mice caused by autoreactive CD4 T cells from Scurfy mice (Sf CD4T cells). Plate bound expanded CD4+CD25+ T cells as in FIG. 8A suppress the wasting diseases.

FIG. 9 shows a bar graph of fold expansion of human CD4 FoxP3+ or CD4+FoxP3− cells by anti-CD3 antibody added to culture or plate immobilized anti-CD3 and anti-CD28 antibody stimulation for 12 days. FoxP3+ cells are shown in open rectangle and FoxP3− cells are shown in solid rectangle. All the cultures contained IL−2 (10 ng/ml).

FIGS. 10A and 10B show dot plots of annexin-V and 7-AAD staining of CD4+CD25− T cells from mouse lacking CD28 (FIG. 10B) or WT control mouse under plate bound stimulation for 5 days (FIG. 10A) in the presence of IL−2. The numbers within the plot represent the percent positive cells in the corresponding quadrant. Only cells in lower left quadrant are live cells, while in lower right are in the process of apoptotic death. Cells in the upper left and right quadrant are dead cells.

FIGS. 11A and 11B show dot plots of annexin-V and 7-AAD staining of CD4+CD25− T cells from a mouse strain lacking P53 (FIG. 11B) or WT control mouse under plate bound stimulation for 5 days (FIG. 10A) in the presence of IL−2. (FIG. 11A). The numbers within the plot represent the percent positive cells in the corresponding quadrant. Only cells in lower left quadrant are live cells, while in lower right are in the process of apoptotic death. Cells in the upper left and right quadrant are dead cells.

FIGS. 12A and 12B show dot plots of annexin-V and 7-AAD staining of CD4+CD25− T cells from control WT mice (FIG. 12A), B6.lpr mice (FIG. 12B), mice lacking Bim (FIG. 12C), P21 (FIG. 12D), TNFR (FIG. 12E) after 5 days of plate bound stimulation in the presence of IL−2. The numbers within the plot represent the percent positive cells in the corresponding quadrant. Only cells in lower left quadrant are live cells, while in lower right are in the process of apoptotic death. Cells in the upper left and right quadrant are dead cells.

FIGS. 13A-D show annexin-V and 7-AAD staining of CD4+CD25− T cells stimulated for 5 days with either plate bound anti-CD3 or plate bound anti-CD3 and anti-CD28 in the presence or absence of IL−2 as indicated. Shown is also the annexin-V and 7-AAD staining of CD4+CD25− T cells stimulated with bead bound anti-CD3 and anti-CD28 (FIG. 13E). Only cells in lower left quadrant are live cells, while in lower right are in the process of apoptotic death. Cells in the upper left and right quadrant are dead cells.

FIGS. 14A-E show dot plots of annexin-V and 7-AAD staining of CD4+CD25− T cells after their stimulation with plate bound anti-CD3 and anti-CD28 in the presence of IL−2 for for indicated amount of time.

### DETAILED DESCRIPTION OF THE INVENTION

### I. Definitions

The term “Tregs” refers to regulatory T cells. Regulatory T cells or Tregs refer to FoxP3+ T cells. The cells are typically mammalian cells, including human T cells. Tregs include CD4+FoxP3+ T cells as well as CD8+FoxP3+ T cells.

The term “effector T cell” refers to FoxP3− T cells. FoxP3− T cells include CD4+FoxP3− and CD8+FoxP3− T cells.

The term “expand” in reference to cell culture refers to an increase in cell population numbers.

The term “effective amount” or “therapeutically effective amount” means a dosage sufficient to decrease T cell activity or to otherwise provide a desired pharmacologic and/or physiologic effect e.g., to induce apoptosis of FoxP3− T cells and expansion of FoxP3+ T cells in vitro or reduce inflammation to an extent to provide relief to subject. The precise dosage will vary according to a variety of factors such as subject-dependent variables (e.g., age, immune system health, etc.), the disease, and the treatment being effected.

The terms “individual,” “host” “subject,” and “patient” are used interchangeably herein, and refer to a mammal, including, but not limited to, mammals, rodents, simians, humans, mammalian farm animals, mammalian sport animals, and mammalian pets.

As used herein, the term “treating” includes alleviating, preventing and/or eliminating one or more symptoms associated with an inflammatory or autoimmune disease.

The term TCR complex refers to the T cell receptor complex. The T cell receptor complex is made up of antigen-recognition proteins and signaling proteins. The antigen-recognition proteins are also known as the T cell receptor and include the α:β heterodimer. The signaling proteins include five signaling chains (γ, δ, ε, ζ and η collectively referred to as CD3.

### II. Methods for Expanding Tregs

It has been discovered that regulatory T cells can be expanded in vitro to produce cultures of T cells that are highly enriched for Tregs. The method advantageously increases the number of FoxP3+ T cells in a cell culture while also decreasing the number of FoxP3− T cells in the culture. It is believed that the method induces or promotes apoptosis of CD4+FoxP3− and CD8+FoxP3− cells (or FoxP3− T cells) which results in the decrease in number of effector T cells in the culture. Reducing the number of effector T cells in the culture is advantageous because effector T cells increase in number quicker than Tregs and will eventually overtake the cell culture if their growth is unchecked. It is believed that the disclosed method for expanding Tregs is the first method that induces apoptosis of effector cells in a T cell culture while expanding the T cell culture to increase the number of Tregs.

One embodiment provides a method for enriching a cell culture for regulatory T cells by culturing T cells on a planar substrate having an effective amount of TCR complex and CD28 ligands attached to the substrate to induce or promote apoptosis of FoxP3− T cells, and to expand FoxP3+ cells, preferably CD4+FoxP3+ cells and CD8+FoxP3+. It will be appreciated that the methods for culturing T cells in vitro are known in the art. See for example Lymphocytes: A Practical Approach, eds Sarah L. Rowland-Jones, Andrew J. McMichael Oxford University Press, 2000.

Lymphocytes can be obtained from a subject, preferably a mammalian subject, even more preferably a human subject. The cells can be sorted to obtain an initial T cell culture to expand. Cell sorting is known in the art and uses devices such as optical flow sorters. Optical flow sorters measure and select user-defined cell types by illuminating individual cells with a laser and detecting the emitted light. The emitted light is spectrally separated (or separated by “color”) to identify the cells of interest. In droplet-based flow sorters, a cell of interest is selected by applying an electrical charge to a fluid stream (containing the sample). An electrically-charged droplet is then produced containing one or more cells. The resulting charged droplet travels through an electric field between two high voltage deflection plates of opposite polarities. These droplets (containing the cells of interest) are eventually deflected into a collection tube for further use.

Other, non-optical cell sorting methods use magnetic fields or differences in particle buoyancy and density. Magnetic cell sorting uses antibody-coated (magnetic) particles that bind to a specific cell type. When the particles pass by a magnetic field, the desired cells are separated. Density gradient cell sorting uses centrifugation to separate desired cells. Other cell sorting methods use sedimentation, affinity adsorption or affinity extraction to select desired cells.

Magnetic cell sorting is frequently used in human therapeutic applications. Magnetic cell sorting methods can occur under aseptic conditions and can supply sufficient cells for therapeutic use.

In one embodiment, a lymphocyte population is obtained from a subject, typically from a blood sample. The blood sample includes a mixture of lymphocytes including Tregs. The cells in the blood sample can be sorted by methods mentioned above, for example based on proteins expressed on the surface of the lymphocytes. Labeled antibodies that bind to CD4, CD25 or a combination thereof can be used to identify and sort Tregs. Such antibodies are commercially available. Once the initial population of cells is obtained, the cell population is expanded in vitro.
A. Culture Surfaces

The initial population of T lymphocytes includes FoxP3− T cells which if not removed, will eventually take over the culture. To enrich the culture for FoxP3+ cells, the mixed lymphocyte population is cultured on conventional planar cell culture substrates or surfaces. The cell culture substrate can be part of a single or multi-well plate, dish, or flask. Preferably, the cell culture substrate is made of glass, plastic, or a non-toxic polymer. Suitable polymers include hydrophobic polymers such as polystyrene.

The culture substrate can be a regular polystyrene or tissue culture treated polystyrene. It will be appreciated that any flat surface that will allow high concentration of antibody attachment via covalent or non-covalent bonds can be used. The choice of coating condition (buffer, pH, volume time and temperature) will depend on chemical and physical property of the culture substrate. Thus, cells will fall down on the surface by gravity and will constantly receive the signal from coated antibody or ligands or any other condition that will allow flat surface or other surface coated ligands to constantly make contact with cells. Suitable cell culture substrates are commercially available. The cell culture substrates are coated with a binding partner specific for the TCR complex and CD28. Preferably, the binding partner is 1) an antibody or antigen-binding fragment thereof against the TCR complex or MHC tetramers loaded with cognate peptides and 2) anti-CD28 antibody or B7 polypeptide or fragment thereof capable of binding to CD28. In one embodiment, the anti-CD28 antibody is agonistic in nature and not superagonistic. These proteins are applied in an amount effective to stimulate FoxP3+ T cell growth and to induce or promote apoptosis in FoxP3− T cells during culture. In one embodiment coated cell culture substrates are produced by incubating the substrates with 5-10 μg/ml each of anti-CD3 and anti-CD28 antibodies in 2 ml volume (0.1 M borate buffer pH 8.5) overnight on 60 mm diameter culture dishes. The plates are washed the following day to remove unbound antibody before the start of the culture.
B. Ligands for TCR Complex
   1. Antibodies to CD3

Ligands that bind to the TCR complex include antibodies, and antigen binding fragments thereof that bind to proteins that together form the TCR complex. In one embodiment, the antibody is specific for a CD3 polypeptide. In preferred embodiments, the antibody agonizes the TCR complex. Anti-CD3 antibodies are known in the art and are commercially available.

The antibodies may be polyclonal or monoclonal. Monoclonal antibodies (mAbs) and methods for their production and use are described in Kohler and Milstein, Nature 256:495-497 (1975); U.S. Pat. No. 4,376,110; Hartlow, E. et al., Antibodies: A Laboratory Manual, Cold Spring Harbor Laboratory Press, Cold Spring Harbor, N.Y., (1988); Monoclonal Antibodies and Hybridomas: A New Dimension in Biological Analyses, Plenum Press, New York, N.Y. (1980); H. Zola et al., in Monoclonal Hybridoma Antibodies: Techniques and Applications, CRC Press, 1982)).

The antibodies may be xenogeneic, allogeneic, syngeneic, or modified forms thereof, such as humanized or chimeric antibodies. Antiidiotypic antibodies specific for the idiotype of an anti-CD3 or anti-CD28 antibody are also included. The term “antibody” is meant to include both intact molecules as well as fragments thereof that include the antigen-binding site and are capable of binding to a same epitope TCR complex and CD28 molecule as intact antibody binds. These include, Fab and F(ab′)₂ fragments which lack the Fc fragment of an intact antibody, clear more rapidly from the circulation, and may have less non-specific tissue binding than an intact antibody (Wahl et al., J. Nuc. Med. 24:316-325 (1983)). Also included are Fv fragments (Hochman, J. et al. Biochemistry 12:1130-1135 (1973); Sharon, J., et al. Biochemistry 15:1591-1594 (1976)). These various fragments are produced using conventional techniques such as protease cleavage or chemical cleavage (see, e.g., Rousseaux et al., Meth. Enzymol., 121:663-69 (1986)).

Polyclonal antibodies are obtained as sera from immunized animals such as rabbits, goats, rodents, etc. and may be used directly without further treatment or may be subjected to conventional enrichment or purification methods such as ammonium sulfate precipitation, ion exchange chromatography, and affinity chromatography. The immunogen may include the complete CD3 or CD28 protein, or fragments or derivatives thereof.

Preferably, the antibodies are monoclonal antibodies. Monoclonal antibodies may be produced using conventional hybridoma technology, such as the procedures introduced by Kohler and Milstein, Nature, 256:495-97 (1975)), and modifications thereof (see above references). An animal, preferably a mouse is primed by immunization with an immunogen as above to elicit the desired antibody response in the primed animal. B lymphocytes from the lymph nodes, spleens or peripheral blood of a primed, animal are fused with myeloma cells, generally in the presence of a fusion promoting agent such as polyethylene glycol (PEG). Any of a number of murine myeloma cell lines are available for such use: the P3-NS1/1-Ag4-1, P3-x63-k0Ag8.653, Sp2/0-Ag14, or HL1-653 myeloma lines (available from the ATCC, Rockville, Md.). Subsequent steps include growth in selective medium so that unfused parental myeloma cells and donor lymphocyte cells eventually die while only the hybridoma cells survive. These are cloned and grown and their supernatants screened for the presence of antibody of the desired specificity. Positive clones are subcloned, e.g., by limiting dilution, and the monoclonal antibodies are isolated.

Hybridomas produced according to these methods can be propagated in vitro or in vivo (in ascites fluid) using techniques known in the art (see generally Fink et al., Prog. Clin. Pathol., 9:121-33 (1984)). Generally, the individual cell line is propagated in culture and the culture medium containing high concentrations of a single monoclonal antibody can be harvested by decantation, filtration, or centrifugation.

The antibody may be produced as a single chain antibody or scFv instead of the normal multimeric structure. Single chain antibodies include the hypervariable regions from an Ig of interest and recreate the antigen binding site of the native Ig while being a fraction of the size of the intact Ig (Skerra, A. et al. Science, 240: 1038-1041(1988); Pluckthun, A. et al., Methods Enzymol. 178: 497-515(1989); Winter, G. et al., Nature, 349: 293-299 (1991)). In a preferred embodiment, the antibody is produced using conventional molecular biology techniques.
2. Major Histocomptability Complex (MIIC)-peptides tetramers

MHC-peptide tetramers, dimer, trimers or multimers can be used as a ligand for the TCR complex. MHC tetramers are based on recombinant MHC class I or MHC class II molecules. To produce MHC class 1-peptide tetramer, MHC class I molecules are folded with the peptide of interest and β2M and tetramerized. To produce MHC class II-peptide tetramer, MHC class II α and β chain are folded with the peptide of interest and tetramerized. This tetramer reagent will specifically bind T cell receptors that are specific for a given peptide-MHC complex. For example, a Kb/FAPGNYPAL tetramer will specifically bind to Sendai virus specific CTL in a C57BL/6 mouse. Antigen specific responses can be measured as CD8+, tetramer+ T cells as a fraction of all CD8+ lymphocytes. The reason for using dimers, tetramers or multimers, as opposed to a single labeled MHC class I or class II molecule is for example that tetrahedral tetramers can bind to four TCRs at once, allowing specific binding in spite of the low (10⁻⁶ molar) affinity of the typical class I-peptide-TCR interaction.
C. Ligands for CD28
   1. Antibodies to CD28

Antibodies and antigen binding fragments thereof can be used as ligands for CD28. The antibodies can be poly clonal or monoclonal. Methods for producing antibodies are well known in the art and are discussed above. Additionally, antibodies specific for CD28 are commerically avaialable.
2. B7 Polypeptides

B7 polypeptides, biologically active fragments thereof, fusion proteins thereof, or a combination of B7 co-stimulatory molecules can also be used as ligands for CD28. Representative B7 polypeptides include, but are not limited to B7-1, B7-2, and combinations thereof. In a preferred embodiment, the extracellular domain of a B7-1, B7-2 or a biologically active fragment thereof is used as a T cell co-stimulatory polypeptide. All or part of the extracellular domain of B7-1 or B7-2 can be used to produce a fusion protein capable of binding CD28.

Variants of co-stimulatory molecules can also be used. Exemplary variants of co-stimulatory molecules are those that have an insertion, deletion, or substitution of one or more amino acids that reduces or prevents the co-stimulatory molecule from participating in signal transduction pathways that transmit inhibitory signals in T cells. In one embodiment, the co-stimulatory molecule is mutated so that it has reduced binding to receptors that transmit inhibitory signals in T cells, for example CTLA4, relative to the non-mutated co-stimulatory polypeptide.

The B7 co-stimulatory polypeptide may be of any species of origin. In one embodiment, the co-stimulatory polypeptide is from a mammalian species. In a preferred embodiment, the co-stimulatory polypeptide is of murine or human origin. Useful human B7 co-stimulatory polypeptides have at least about 80, 85, 90, 95 or 100% sequence identity to the B7-1 encoded by the nucleic acid having GenBank Accession Number NM_{—}005191; or the B7-2 polypeptide encoded by the nucleic acid having GenBank Accession Number U04343. Certain embodiments provide compositions including CD28 binding extracellular domain of B7 co-stimulatory. Such extracellular domains have at least about 80, 85, 90, 95 or 100% sequence identity to the extracellular domains of the polypeptides encoded by the nucleic acids having GenBank Accession Numbers NM_{—}005191 or U04343. Typically the signal sequence of the polypeptide is removed.
3. B7 fusion polypeptides

B7-1 and B7-2 polypeptides or fragments thereof may be coupled to other polypeptides to form fusion proteins that also serve as ligands to CD28. Representative B7 co-stimulatory fusion polypeptides have a first fusion partner including all or a part of a B7 protein or B7 variant polypeptide fused (i) directly to a second polypeptide or, (ii) optionally, fused to a linker peptide sequence that is fused to the second polypeptide. The presence of the fusion partner can alter the solubility, affinity and/or valency of the B7 polypeptide. As used herein, “valency” refers to the number of binding sites available per molecule. The B7 fusion proteins described herein include any combination of amino acid alteration (i.e. substitution, deletion or insertion), fragment of B7, and/or modification. In one embodiment, variant B7 fusion proteins include the extracellular domain of a B7-1, or B7-2, or a CD28 binding fragment thereof, as the first binding partner. In another embodiment, variant B7 fusion proteins include the IgV and IgC domain of a B7 protein as the first binding partner. In another embodiment, variant B7 fusion proteins include the IgV domain of a B7 protein as the first binding partner.

The second polypeptide binding partner may be N-terminal or C-terminal relative to the B7 polypeptide or variant B7 polypeptide. In a preferred embodiment, the second polypeptide is C-terminal to the B7 polypeptide or variant B7 polypeptide.

A large number of polypeptide sequences that are routinely used as fusion protein binding partners are well known in the art. Examples of useful polypeptide binding partners include, but are not limited to, green fluorescent protein (GFP), glutathione S-transferase (GST), polyhistidine, myc, hemagglutinin, Flag™ tag (Kodak, New Haven, Conn.), maltose E binding protein and protein A. In one embodiment, the variant B7 fusion protein is fused to one or more domains of an Ig heavy chain constant region, preferably having an amino acid sequence corresponding to the hinge, C_{H2} and C_{H3} regions of a human immunoglobulin Cγ1 chain or other human immunoglobulin Fc portions.
D. Cell Culture Conditions

The regulatory T cells are expanded in vitro using conventional lymphocyte cell culture techniques and conditions. For example, the cells are cultured on the coated culture substrates in a humidified chamber at 37° C. in the presence of 5.0% CO₂. The cells can be cultured for several days. Typically the cells are cultured for one to 14 days or more.

Suitable media for culturing the Tregs includes but is not limited to RPMI-1640 medium. The medium can optionally be supplemented with growth factors, cytokines, antibiotics, co-factors, and other supplements known in the art. For example the medium can be supplemented with 1 to about 20%, typically about 5 to about 10% fetal calf serum (FCS) or other alternative to FCS for the safety of the patient. Reducing factors such as 2-mercaptoethanol can also be added to the medium, for example at about 50 μM. Buffers for maintaining the pH can also be added. Suitable pH buffers include, but are not limited to HEPES. The concentration of HEPES or other pH buffer is typically from about 10-25 mM. Additional additives such as sodium pyruvate, amino acids, for example MEM non essential amino acids solution ˜1X and MEM non-essential amino acid solution˜0.5X can be added.

Representative cytokines that can be added to the medium include, but are not limited to interleukin−2 (IL−2). Representative concentrations of IL−2 that can be used include from about 1 to about 20 ng/ml, typically about 10 ng/ml. IL−2 is commercially available.

It will be appreciated that the culture conditions can be modified to include equivalent buffers, supplements, and additives routinely used when culturing lymphocytes. For example, serum-free media can be used to replace the use of FCS. Replacing FCS will be more desirable for expansion of Tregs for clinical purposes.

The cultures are spilt onto newly coated plates when density of cells reaches at ˜0.5−1.0×10⁶ cells/ml. The cultures are terminated when desired numbers of FoxP3+ T cells are obtained.
E. Antigen Specific Expansion

One embodiment provides a method for expanding antigen specific regulatory T cells. Antibodies are not the natural ligands for TCR complex and CD28. Therefore, natural ligands for TCR complex and CD28 can replace the use of antibody during Treg expansions. The natural ligand for CD28 includes intact B7.1 or B7.2 molecules (mammalian origin). Alternatively, B7.1 or B7.2 fused to Fe portion of immunoglobulin (Ig) (B7.1 or B7.2) (B7.1-Ig or B7.2Ig) may be used to deliver signal through CD28. The immunoglobulin will be preferably from human origin, but may include any mammalian lg. The nucleotide and amino acid sequence for B7 proteins are known in the art and are available for example from GenBank and other similar databases.

The TCR complex binds to major histocomptability complex (MHC)-loaded with cognate peptide (MHC-peptide). Soluble MHC-peptide complexes are produced as monomer, dimmers, tetramer or pentamers. They are commercially available or are available from National Institute of Health (NIH) tetramer core facility.

For expansion of antigen specific Tregs anti-CD3 antibody can be replaced with MHC-bound to a cognate peptide. MHC molecules (HLA in humans and H-2 in mouse) are of same origin as of the subject or patients. Cognate peptide is derived from one or more specific antigens in question. The antigen will be derived from (but not limited to) tissues/organs against which immune responses have to be suppressed. In some cases these peptides are modified to increases its binding to MHC or increase activation of T cells. These are referred as agonistic peptides or altered peptide ligand or mimotopes or epitopes.

For use of Tregs in improving the longevity of transplants, the MHC will be of donor origin (organ donor), while the initial source of Tregs will be from the recipient. In this case, the peptides will be derived from different antigens from either the recipient or donor based on their ability to bind to donor MHC and elicit a T cell response. Also, in cases of transplant rejection, MHC can be loaded with more than one peptide].

In another embodiment, the cells are cultured in the absence of rapamycin.

### III. Treg Cell Cultures

Cultures of regulatory T cells produced using the disclosed methods are enriched for FoxP3+ cells. In certain embodiments, the culture is enriched for FoxP3+ by at least about 50, 100, 150, 200, 250, 300, 350, or 400 fold. In a preferred embodiment, the culture is enriched for FoxP3+ cells by more than 100 fold. Another embodiment provides cultures of regulatory T cells that contain less than about 20%, 15%, 10% FoxP3− T cells after about 1 to about two weeks, preferabaly after about 2 to 12 days, even more preferably after about 6 to 8 days.

Prior the disclosed methods for expanding Tregs, earlier methods could not expand Tregs in high purity (>80% FoxP3+) if cultures were started with either CD4+ or CD4+CD25+ T cells, because contaminating FoxP3− T cells overtake the culture. To expand the Tregs in high purity with previous methods more than 98% pure CD4+CD25+CD62L+ positive population or addition of rapamycin is needed. When total CD4+ T (˜10% FoxP3+ in the beginning) cells are cultured with CD3 and CD28 coated beads in the presence of rapamycin, after 3 weekly cycles only ˜30% of the cells are FoxP3+. On the other hand with the disclosed technique, when total CD4+ T cells are cultured after ˜8 days of culture >80% of the cells are FoxP3+. The rapamycin also suppresses the expansion of FoxP3+ T cells, albeit at a much lesser extent than FoxP3− T cells (Basu S, J. Immunol, 180:5794 (2008)).

The expanded T reqs can by cryogenically stored or preserved. Typically, the cells are concentrated to about 5×10⁶ to 15×10⁶ cells/ml and frozen in cryogenic storage tubes. The cells are typically frozen in about 90% (v/v) FCS and 10% of a cryopreservative agent such as dimethyl sulfoxide (DMSO). DMSO can be used from about 1 to about 15% (v/v).

### IV. Methods of Using Tregs

The enriched cultures of Tregs can be used in adoptive T cell therapy to treat one or more symptoms associated with inflammation or an autoimmune disorder. One embodiment provides a method for adoptive T cell transfer to treat one or more symptoms associated with an inflammatory disorder or an autoimmune disorder.
A. Diseases and Disorders to be Treated

Disease and disorders that can be treated using the disclosed compositions and methods include, but are not limited to inflammatory disorders and autoimmune disorders. Representative diseases and disorders that can be treated include type 1 diabetes, multiple sclerosis, rheumatoid arthritis, inflammatory bowel disease, systemic lupus erythematosus, graft-versus-host disease, various kinds of graft/transplant rejection, allergies and harmful inflammations during infections.

Another embodiment provides a method for treating one or more symptoms of an inflammatory or autoimmune disease or disorder including administering to a subject an effective amount of the Treg cells obtained by the disclosed methods to inhibit or reduce an immune response in the subject. One embodiment provides a method for treating one or more symptoms of an inflammatory disorder or autoimmune disorder by obtaining a blood sample from a subject. The blood sample typically includes a mixed population of lymphocytes. The term “mixed populations of lymphocytes” refers to a population of lymphocytes containing both Tregs and other T cells, in particular effector T cells. The mixed population of lymphocytes is sorted to enrich for T cells, preferably Tregs. Once an initially enrich culture is obtained the culture is expanded as described above. The Tregs can be further selected by culturing them in the presence of specific antigens.

Once a desired population of Tregs is obtained they are administered to the subject. The expanded Tregs can be parenterally administered to a subject in need thereof Typically, the cells are administered by injection or infusion. Expanded Tregs are preferably antigen specific Tregs and can be administered to a subject repeatedly over a period of days, weeks or months. In one embodiment, about 10-100 million or more Tregs are administered. The frequency may be biweekly or monthly.

The expanded Tregs are administered in an amount effective to inhibit or reduce an immune response in the subject in order to improve his/her health. The term “immune response” includes T cell activation or T cell activity or T cell function or inflammation mediated destruction of tissue.
B. Combination Therapy

The disclosed cells can be administered to a subject in need thereof alone or in combination or following one or more additional therapeutic agents including, but not limited to immunosuppressive agents, e.g., antibodies against other lymphocyte surface markers (e.g., CD40) or against cytokines, other fusion proteins, e.g., or other immunosuppressive drugs (e.g., steroids), anti-proliferatives, cytotoxic agents, or other compounds that may assist in immunosuppression. It will be appreciated that compounds that inhibit the survival or suppressive ability of Tregs will not be combined with the disclosed expanded Treg cultures.

As used herein the term “rapamycin compound” includes the neutral tricyclic compound rapamycin, rapamycin derivatives, rapamycin analogs, and other macrolide compounds which are thought to have the same mechanism of action as rapamycin (e.g., inhibition of cytokine function). The language “rapamycin compounds” includes compounds with structural similarity to rapamycin, e.g., compounds with a similar macrocyclic structure, which have been modified to enhance their therapeutic effectiveness. Exemplary Rapamycin compounds are known in the art (See, e.g. WO95122972, WO 95116691, WO 95104738, U.S. Pat. Nos. 6,015,809; 5,989,591; U.S. Pat. Nos. 5,567,709; 5,559,112; 5,530,006; 5,484,790; 5,385,908; 5,202,332; 5,162,333; 5,780,462; 5,120,727).

The language “FK506-like compounds” includes FK506, and FK506 derivatives and analogs, e.g., compounds with structural similarity to FK506, e.g., compounds with a similar macrocyclic structure which have been modified to enhance their therapeutic effectiveness. Examples of FK506-like compounds include, for example, those described in WO 00101385. Preferably, the language “rapamycin compound” as used herein does not include FK506-like compounds.

Other suitable therapeutics include, but are not limited to, anti-inflammatory agents. The anti-inflammatory agent can be non-steroidal, steroidal, or a combination thereof. One embodiment provides oral compositions containing about 1% (w/w) to about 5% (w/w), typically about 2.5% (w/w) or an anti-inflammatory agent. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, nifiumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Unless defined otherwise, all technical and scientific terms used herein have the same meanings as commonly understood by one of skill in the art to which the disclosed invention belongs. Publications cited herein and the materials for which they are cited are specifically incorporated by reference.

### V. Implants

Another embodiment provides an implant for increasing the number of FoxP3+ T cells in a subject. The implant includes a substrate coated with binding partners to the TCR complex and CD28 and allows sustained interaction of T cells with the coated substrate.

The substrate of the implant can be made of metal, ceramic, synthetic polymers, natural polymers, or combinations thereof. In certain embodiments, the implant is biodegradable or bioabsorbable. In one embodiment, the substrate is made from a metal or metal alloy. Representative metals include, but are not limited to titanium, silver, gold, or combinations thereof.

In a preferred embodiment, the substrate is made of one or more polymers. Typically, the substrate is a matrix. Representative natural polymers include, but are not limited to collagen, fibrinogen, polysaccharides, proteins, gelatin, and combinations thereof. In one embodiment, the polymer is a polyhydroxyalkanoate or copolymer thereof. Representative polymers include, but are not limited to polyalkylene esters, polylactic acid and copolymers thereof, polyamide esters, polyvinyl esters, polyvinyl alcohol, polyanhydrides, and combinations thereof.

Preferred polymers for bioabsorbable implants are poly-L-lactic acid (PLLA), polyglycolic acid (PGA), poly (D, L-lactide/glycolide) copolymer (PDLA), and polycaprolactone (PCL).

The implants may include additives such as plasticizers, antioxidants, pigments and stabilizers.

In certain embodiments, the implants are in the shape of a rod, cylinder, film, disk or microparticles. The polymer may be cast as a thin slab or film, ranging from nanometers to four centimeters.

The implants can be formed using conventional techniques, including for example solvent evaporation, spray drying, solvent extraction and other methods known to those skilled in the art.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1: Plate-bound anti-CD3/anti-CD28 favors proliferation of CD4+CD25+ T cells.

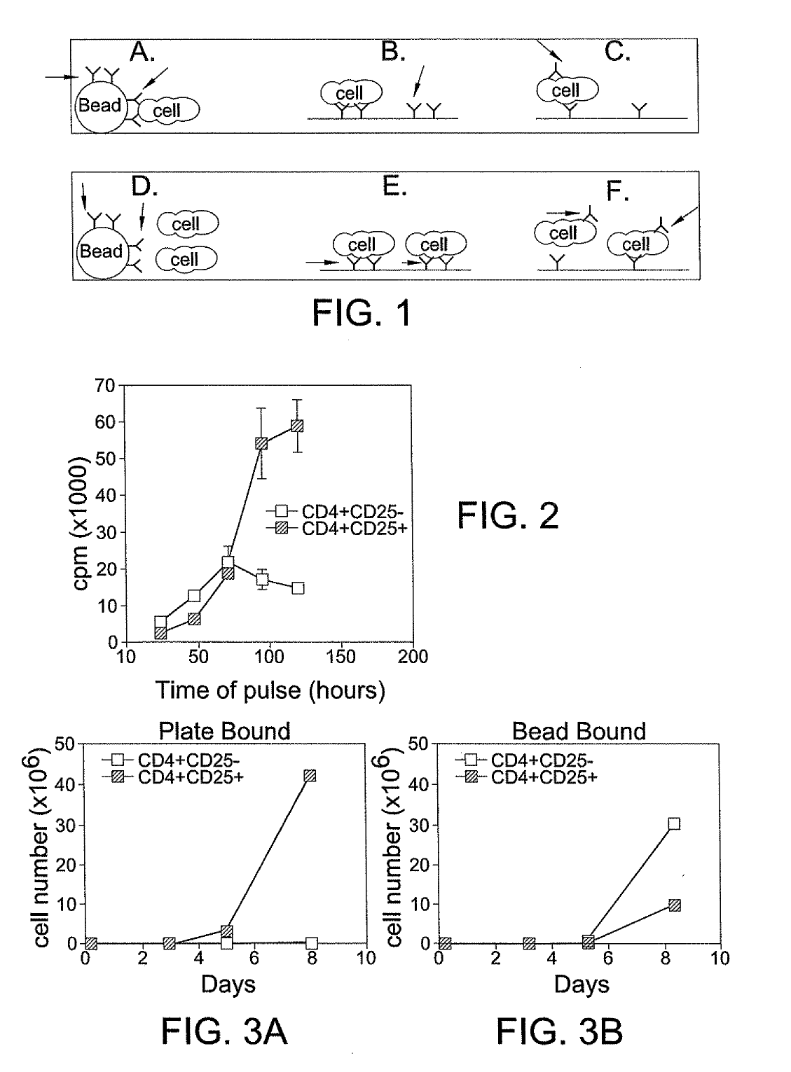

Stimulation of sorted nTregs (CD4+CD25+ T cells) by beads coated anti-CD3 and anti CD28 antibodies (referred as ‘bead bound’ hereafter) has been used as an effective procedure to expand nTregs. During bead bound stimulation, after ˜3-4 days, T cells start leaving the beads after some time of stimulation and were no longer receiving activation signal through anti-CD3 and anti-CD28 (FIG. 1A). In other routinely use practice, CD28 costimulation of plate bound anti-CD3 stimulated T cells in vitro cultures is provided by addition of anti-CD28 in the medium that may lead to ligation of CD3 and CD28 at different locations in the cells (FIG. 1C). In order to provide ligation of CD3 and CD28 close to each other and for sustained period of time, anti-CD3 and anti-CD28 antibody were immobilized to the plates at high density (referred as ‘plate bound’ hereafter) (FIG. 1B).

The proliferation of T cells induced by the plate bound method was investigated. Sorted CD4+CD25− and CD4+CD25+ T cells (1500/well, flat bottomed 96 well plate) were stimulated with plate bound anti-CD3/anti-CD28 and their proliferation was measured by pulsing cells with [³H]thymidine at 24, 48, 72, 96 and 120 hours after activation for 12 hours. All the cultures were supplemented with 10 ng/ml of IL−2. Rate of proliferation was comparable between CD4+CD25− and CD4+CD25+ cells for the first 72 hours (FIG. 2). However, CD4+CD25− T cells proliferation declined after 3 days as judged by their uptake of [³H]thymidine. In contrast, ³H-thymidine uptake increased ˜>2.5 fold by CD4+CD25+ between 72-96 hours, while there was almost no increase between 96-120 hours. No increase in proliferation of CD4+CD25+ T cells from 96-120 hours means either cells stopped proliferation or nutrients went limiting because proliferation was setup in small volume (200 μl/well).

### Example 2: Plate Bound Stimulation Induces Apoptosis of CD4+CD25− (FoxP3−) T cells and Better Expansion of CD4+CD25+ T cells. And

To rule out the nutrient depletion possibility in FIG. 1, the stimulation of 10⁵ CD4+CD25− and CD4+CD25+ T cells in 5 ml medium in 60 mm plates either by plate bound or by bead bound stimulation in the presence of 10 ng/ml of IL−2 and cell were analyzed for live cell number and FoxP3 expression. On day 5 cultures were split (1:6) onto newly anti-CD3 and anti-CD28 coated plates. After 8 days of culture with plate bound stimulation, the number of CD4⁺CD25⁺T cells increased by 420 fold whereas CD4⁺CD25⁻T cell number increased only by 5 folds (FIG. 3A). When these cells were stimulated by bead bound stimulation, more robust growth of CD4+CD25− T cells (250 fold increase) was observed than CD4+CD25+ T cells (90 fold increase) (FIG. 3B).

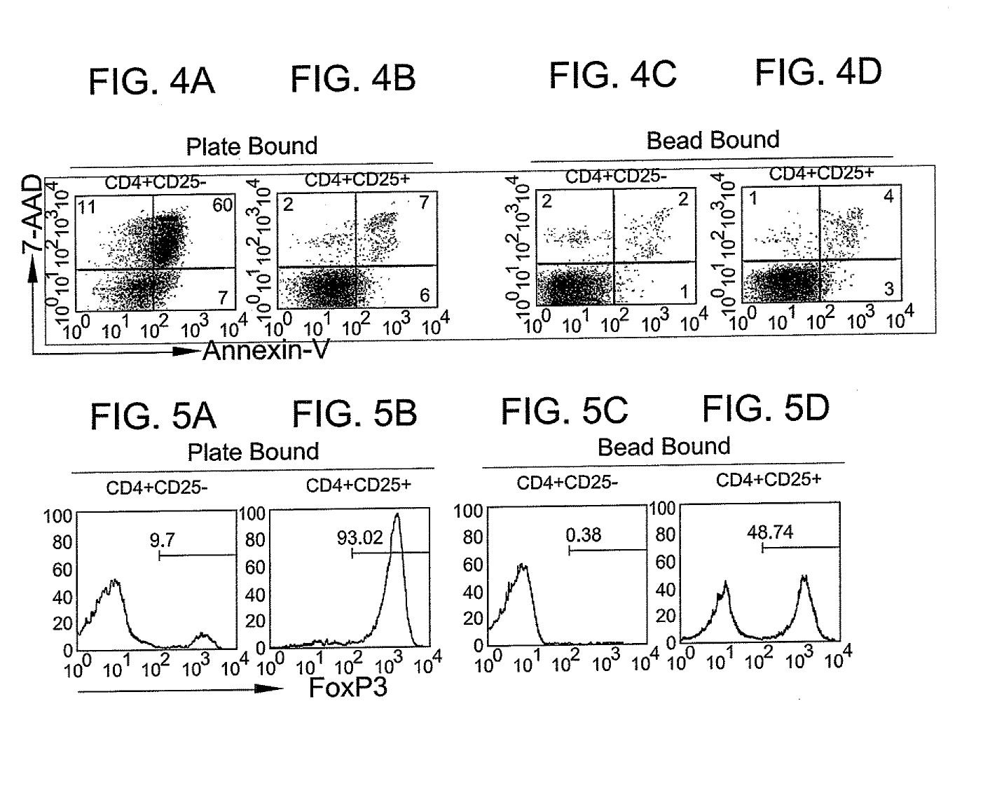

At around 4-5 days, cells in the plate bound stimulated CD4+CD25− cultures appeared dead under microscope. To quantify the cell death, cells from these conditions were analyzed for viability by staining with annexin-v and 7-AAD. In bead bound stimulated CD4+CD25− and CD4+CD25+ and plate bound stimulated CD4+CD25+ cultures, there was a very minimal cell death (4%, 5% and 9% cells were positive 7-AAD, respectively), while majority of cells (>85%) were live (annexin-V−7-AAD-) (FIG. 4B-D). In sharp contrast, in plate bound stimulated CD4+CD25− cultures, 71% of the cells were dead (AAD+), while 7% cells showed early signs of apoptosis (annexin-V+7-AAD-) and only ˜22% of the cells were live (annexin-V−7-AAD-) (FIG. 4A). Mouse CD8+ T cells (FoxP3−) also underwent apoptotic cell death after plate bound stimulation (data not shown).

### Example 3: Effector T cells (FoxP3−) Predominate in long term expanded bead bound expanded CD4+CD25+ cultures and not in plate bound expanded CD4+CD25+ cultures.

The majority of cells in plate bound and bead bound stimulated CD4+CD25− cultures remain FoxP3− (>99% and >90% respectively) (FIG. 5A and 5C). After 8 days of culture, in bead bound stimulated CD4+CD25+ cultures only 48.74% cells expressed FoxP3 (FIG. 5D), while 93.02% cells (FIG. 5B) in plate bound stimulated CD4+CD25+ cultures were positive for FoxP3. After 10 days of stimulation of CD4+CD25+ T cells by bead bound and plate bound stimulation 12% and 89% of the cells were FoxP3+ (data not shown). It is important to note that same initial seeds of CD4+CD25+ and CD4+CD25− T cells were used for expansion in bead bound and plate bound stimulation. Thus, bead bound stimulated cultures were progressively dominated by FoxP3− (effector) T cells, while plate bound stimulated cultures maintained FoxP3 positive cells with time.

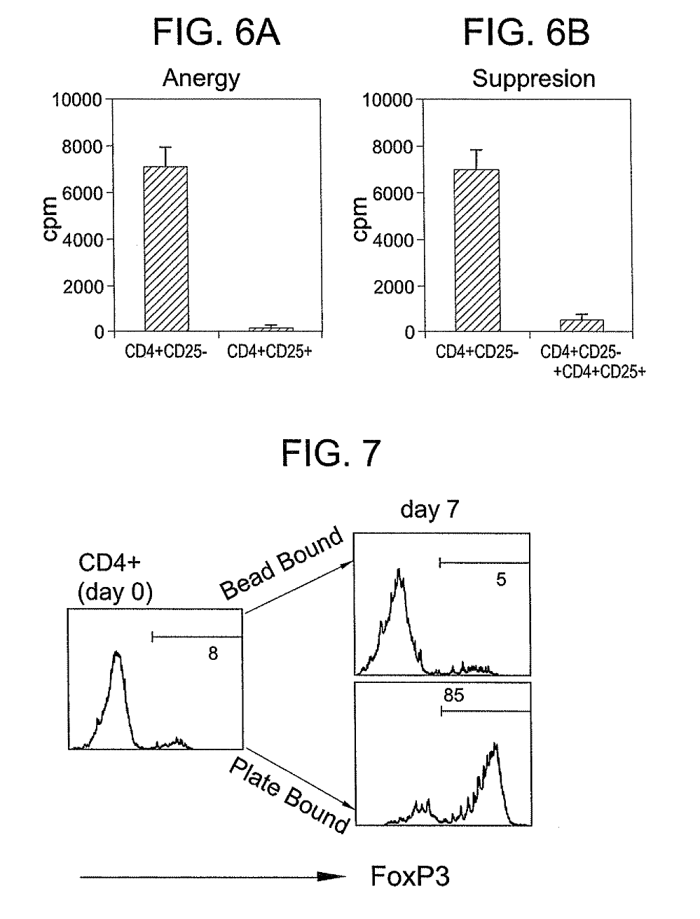

Naturally arising regulatory T cells are FoxP3 positive, anergic to anti-CD3 mediated proliferation and suppress the proliferation of CD4+CD25− T cells in vitro. It is important that plate bound expanded Treg preserve their function. Indeed plate bound expanded Treg did not exhibit anti-CD3 proliferation (FIG. 6A) and suppressed the proliferation of freshly isolated CD4+CD25− T cells (FIG. 6B).

These data demonstrate that plate- bound antibody stimulation is different from bead bound antibody stimulation in 2 respects 1) It expands CD4+CD25+ (mostly FoxP3+) T cells in more numbers 2) it induces apoptosis of CD4+CD25− (largely FoxP3−) cells.

### Example 4: Plate bound not bead bound expanded CD4 T cells are enriched in FoxP3+ cells.

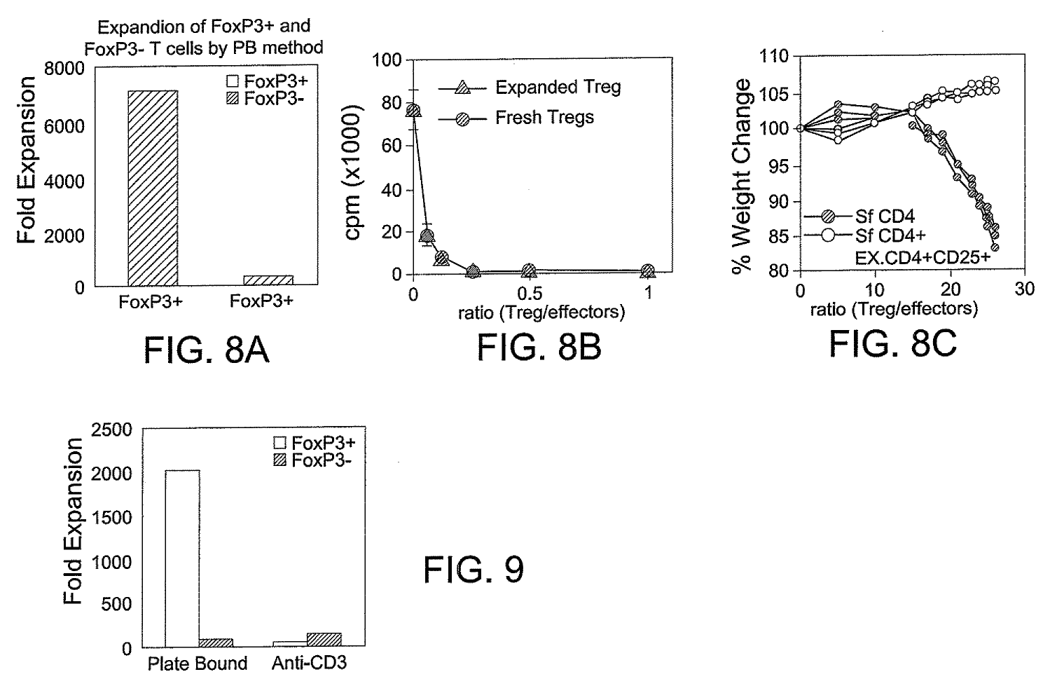

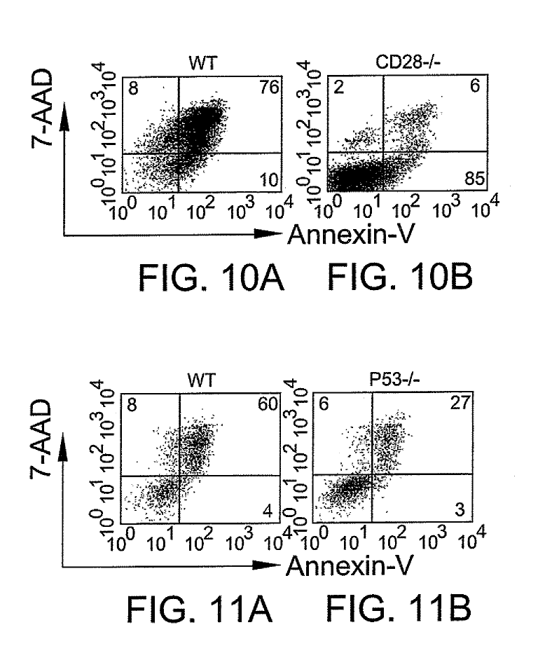

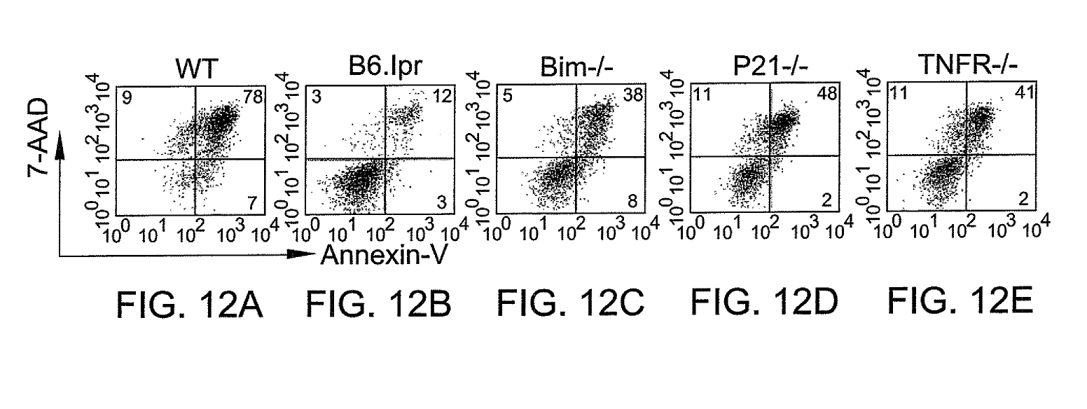

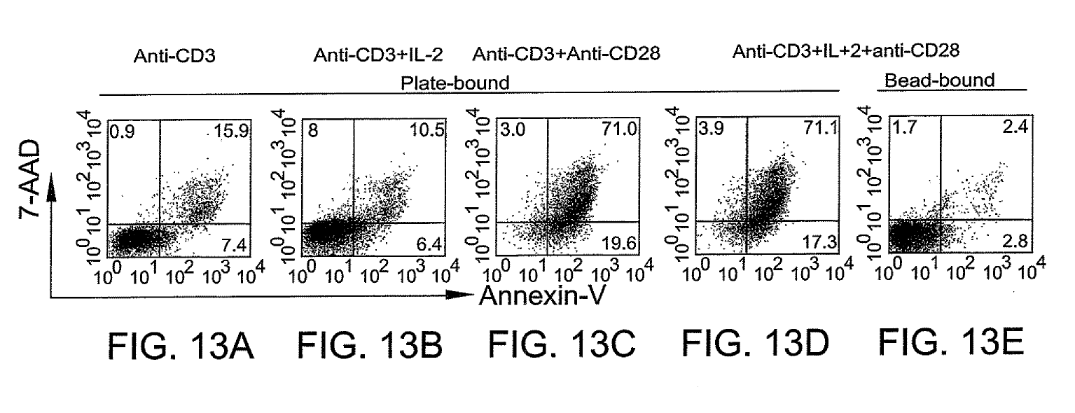

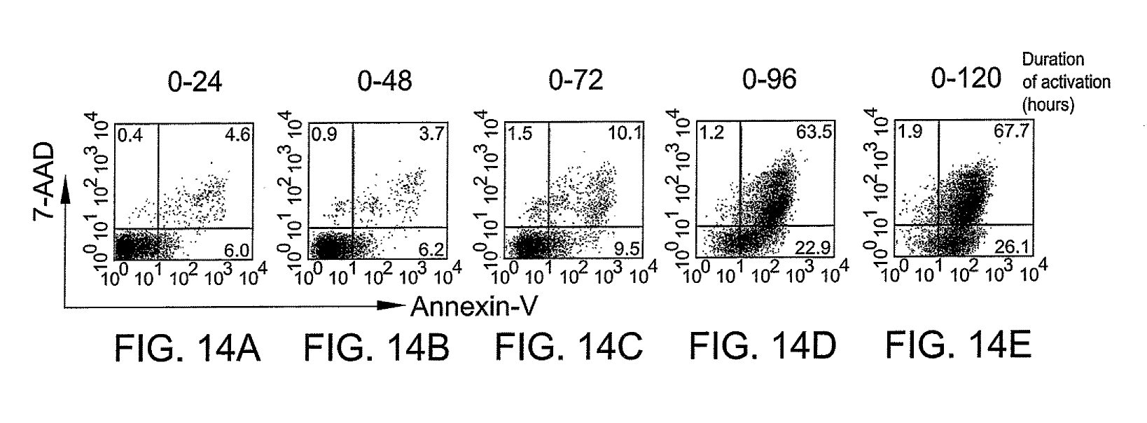

The above data (FIGS. 3-5) suggest that if total CD4 T cells are cultured with plate bound stimulation, FoxP3− T cells will undergo apoptosis, while FoxP3+ cells will survive and expand. Thus, CD4 T cells expanded by plate bound stimulation will be enriched for FoxP3+ cells. However, this will not be case with bead bound expanded CD4 T cells. CD4+ T cells (initial 8% FoxP3+) were expanded by bead bound and plate bound stimulation. After 7 days of expansion with plate bound and bead bound stimulation 85% and 5% of the CD4+ T cells were FoxP3+ respectively (FIG. 7). Thus, even total when initial seed of CD4 contains minority of FoxP3+ cells (a condition that may be more common working with human cells, or when sorting efficiency was not optimal), plate bound stimulation was able to selectively expand FoxP3+ T cells. Plate bound expanded Tregs from initial pool of total CD4+ T cells are functional. CD4+CD25+ T cells expanded from total CD4+ T cells by plate-bound stimulation potently suppressed the proliferation of CD4+CD25− T cells induced by anti-CD3 antibody (FIG. 8B) and this suppression was comparable to that exhibited by freshly isolated CD4+CD25− T cells. Also, plate bound expanded CD4+CD25+ T cells suppresses the wasting syndrome and weight loss in Rag1−/− mice caused by auto reactive T cells from autoimmune Scurfy mice (FIG. 8C). These data are consistent with use of plate bound expanded cells for therapeutic use to suppress autoimmune disorders and inflammations.

The data in FIGS. 7-8 suggest that if total CD4 T cells were cultured by plate bound stimulation, the expanded population will be dominated by FoxP3+ cells (Tregs). Next, human lymphocytes were expanded by plate bound stimulation. On day 0, 90.4% and 9.6% of the CD4 T cells were FoxP3− and FoxP3+ respectively (data not shown). After 12 days of culture with plate bound anti-CD3 and anti-CD28, there was 2015 and 68 fold expansion of FoxP3+ and FoxP3− CD4+ T cells respectively in the presence of IL−2 (FIG. 9). Over the same period anti-CD3 (cross linked to antigen presenting cells) and IL−2 mediated stimulation expanded FoxP3+ and FoxP3− CD4 T cells by 56 and 126 folds respectively (FIG. 9). The staining for FoxP3 was performed using antibody clone 259D, which does not have any known cross reactivity to other proteins. Plate bound expanded cells suppressed the proliferation of syngenic lymphocytes induced by anti-CD3 (data not shown). Collectively these data suggest that plate bound stimulation can be used for expansion of human FoxP3+ T cells for clinical purposes.

1. A method for expanding regulatory T cells comprising
culturing a population of T cells on a substrate, wherein the population of T cells comprises FoxP3− and FoxP3+ T cells, and wherein the substrate comprises binding partners for the TCR complex and CD28 attached to the substrate in an amount effective to expand FoxP3+ T cells and promote apoptosis of FoxP3− T cells.

2. The method of claim 1 wherein the FoxP3− T cells are selected from the group consisting of CD8+ and CD4+CD25− cells.

3. The method of claim 1 wherein the Fox3P3+ T cells are selected from the group consisting of CD4+ cells, CD8+ cells, and combinations thereof.

4. The method of claim 1 wherein the population of cells is cultured in vitro.

5. The method of claim 1 wherein the binding partners for TCR complex and CD28 are independently selected from the group consisting of antibodies or fragments of antibodies that bind the TCR complex or CD28.

6. The method of claim 1 wherein the binding partners for CD28 are selected from the group consisting of B7.1, B7.2, B7.1-Ig, B7.2-Ig, a fusion protein containing CD28 binding portion of B7.1 or B7.2 and anti-CD28 antibody.

7. The method claim 1 wherein the binding partner for TCR complex is selected from the group consisting of anti-CD3 antibody, anti-TCR-β antibody and MHC-peptide dimmer, multimers or tetramers.

8. The method of claim 1 wherein the FoxP3+ cells are expanded by more than 200 fold.

9. The method of claim 1 wherein the FoxP3+ cells are expanded by more than 300 fold.

10. The method of claim 1 wherein the FoxP3+ cells are expanded by more than 400 fold.

11. The method of claim 1 wherein the regulatory T cells are mammalian.

12. The method of claim 1 wherein the regulatory T cells are human.

13. The method of claim 1 wherein the binding partners for the TCR complex and CD28 are covalently attached to the substrate.

14. The method of claim 1 wherein the binding partners for the TCR complex and CD28 are non-covalently attached to the substrate.

15. The method of claim 1 wherein the substrate is planar.

16. The method of claim 1 wherein population of cells make sustained contact with ligands for the TCR complex and CD28.

17. A method for enriching a T cell culture with FoxP3+ cells comprising
culturing a heterogeneous population of T cells comprising FoxP3+ T cells and FoxP3− T cells on a substrate having binding partners for the TCR complex and CD28 attached to the substrate in an amount effective to expand FoxP3+ T cells and promote apoptosis of FoxP3− T cells.

18. The method of claim 17 wherein the binding partners for TCR complex and CD28 are independently selected from the group consisting of antibodies, fragments of antibodies that bind to the TCR complex or CD28.

19. The method of claim 17 wherein the FoxP3+ T cells are expanded by more than 400 fold.

20. A cell culture vessel comprising a substrate, wherein ligands for the TCR complex and CD28 are attached to the substrate in an amount effective to expand FoxP3+ T cells and promote apoptosis of FoxP3− T cells.

21. The cell culture vessel of claim 20 wherein the substrate is substantially planar.

22. The cell culture vessel of claim 21 wherein the ligands for TCR complex or CD28 are covalently attached to the substrate.

23. The cell culture vessel of claim 20 wherein the ligands for TCR complex or CD28 are non-covalently attached to the substrate.

24. The cell culture vessel of claim 20 wherein the FoxP3+ and FoxP3− T cells are mammalian.

25. The cell culture vessel of claim 20 wherein the FoxP3+ and FoxP3− T cells are human.

26. A method for treating one or more symptoms of an inflammatory or autoimmune disease or disorder comprising administering to a subject an effective amount of the cells obtained by the method of claim 1 to inhibit or reduce an immune response in the subject.

27. A culture of regulatory T cells obtained by the method of claim 1.

28. The culture or regulatory T cells of claim 27 wherein the culture contains less than 20% effector T cells after at least fives days of culture.

29. An implant comprising a substrate, wherein the substrate comprises binding partners selected from the group consisting of TCR binding partners, CD28 binding partners, or a combination thereof in an amount effective to induce apoptosis of FoxP3− T cells and increase numbers of FoxP3+ T cells,

30. The implant of claim 29 wherein the substrate comprises a polymer.

31. The implant of claim 30 wherein the polymer is biodegradable.
